(19) United States
(12) Patent Application Publication
(10) Pub. No.: US 2019/0038450 A1
(43) Pub. Date: Feb. 7, 2019

(54) SHOULDER COMPRESSION HARNESS

(71) Applicant: Donald W. Thompson, Columbia, SC (US)

(72) Inventor: Donald W. Thompson, Columbia, SC (US)

(21) Appl. No.: 15/665,518

(22) Filed: Aug. 1, 2017

(51) Int. Cl. A61F 5/02

(52) U.S. Cl. CPC A61F 5/024; A61F 5/026

(57) ABSTRACT

A shoulder compression harness includes a single elastic strap formed into a figure 8 configuration. The intersection of the elastic strap is affixed to itself, so that where the strap crosses itself, that intersection is fixed in position in any suitable manner, in order to provide two generally equal size shoulder loops. The shoulder loops are designed to fit over a wearer's shoulders, and each shoulder loop includes a handle strap, or the like, on a front portion thereof, so that a wearer may adjust the position of each shoulder loop around the shoulder so that it fits comfortably and provides proper posture. When the shoulder compression harness is properly worn, the intersection, or “hub,” is disposed at a central portion of the wearer's back, between the shoulder blades, and also includes a handle for adjustment purposes.

### TECHNICAL FIELD

The invention relates generally to devices used for helping people to develop and maintain proper posture, particularly in the upper body and shoulder areas. More specifically, the present invention relates to compression harnesses that are worn over a wearer's shoulders to urge the shoulders back, and to provide proper posture to the wearer, either during exercise sessions, or during normal daily activities.

### BACKGROUND OF THE INVENTION

In modern times, the average person spends much of their day working at a computer, looking at a cell phone, or driving in traffic, and these normal activities contribute to many different health problems associated with slouching and poor posture. Entire industries have arisen to address posture problems, including ergonomic office furniture, a resurgence of standing desks, and many other devices that are designed to help a person develop and maintain proper posture while working or performing normal, everyday activities.

Many different types of shoulder harnesses and braces have been developed for various purposes. The following references are hereby incorporated herein by reference in their entirety:

U.S. Pat. No. 3,382,868—Universal Clavicle Splint

An orthopedic clavicle brace which applicable to patients regardless of body size and which can be removed and replaced by the patient or another without loss of the size adjustments provided at the original professional fitting. An adjustable-length and flexible but inextensible connector strap lies along the upper spine, and from its upper end diverge respective adjustable length straps which encircle the shoulders and armpits and connect detachably by hooks to a ring at the lower end of the connector strap. These diverging straps are slidably enclosed in soft tubular protectors which can be cut to fit according to patient size.

U.S. Pat. No. 3,548,818—Shoulder Brace

A shoulder brace having a back panel made of elastic webbing stretchable vertically and a pair of shoulder straps made of elastic webbing stitched at their ends to the top and bottom of the back panel and which extend about the shoulders when worn. The shoulder straps are stretchable in a body encircling direction, and each is provided with an Helanca lining which does not interfere with the stretch of the straps.

U.S. Pat. No. 3,718,137—Clavicle Brace

A clavicle brace for supporting the shoulder region of the body in a fixed position without biting into the body of the wearer. The brace comprises a back strap and a pair of upwardly extending shoulder straps connected at the upper end of the back strap. The shoulder straps include a contoured, non-biting, porous core of a resilient foam material which distributes the forces from the shoulder straps over a broad area of contact. Since the core is porous, ventilation to the surface of the skin beneath the straps is permitted.

U.S. Pat. No. 3,856,004—Clavicle Brace

A clavicle brace for giving support to a patient's shoulders during the healing of an injured clavicle. The clavicle brace includes a body portion and integral first and second strap portions made generally of reticulated foam. A coupling means is substantially fixedly attached to the body portion for allowing the distal ends of the first and second strap portion to extend there around after passing over the patient's shoulders and under his axillae. The distal ends of the first and second strap portions are then doubled back on themselves and attached by Velcro type fastening means to other parts of the first and second strap portions thereby properly securing the clavicle brace to the patient.

U.S. Pat. No. 3,897,776—Clavicle Splint

A clavicle splint for supporting the shoulder region of the body in a fixed position and which comprises a vertically directed back strap, and a pair of upwardly extending shoulder straps connected at the upper end of the back strap. Each shoulder strap comprises a porous core of resilient foam material having a semicircular cross-sectional configuration which closely conforms to the anatomy of the wearer and thereby reduces the tendency of the straps to bite into the body of the wearer. Also, the core is arcuately curved along the longitudinal length thereof so as to tend to naturally encircle the shoulder and axilla and thereby alleviate the tendency of the core to buckle upon being positioned on the body of the wearer.

U.S. Pat. No. 4,570,619—Clavicle Brace

A shoulder brace of the type having a generally figure-8 configuration with right and left shoulder loops. Each of the shoulder straps that define the brace's right and left loops are provided with a cushioned inner surface adapted to extend over the wearer's trapezius muscle, over the anterior surface of the wearer's shoulder, and under the wearer's axilla so that the strap does not tend to bite into the wearer's body when the brace is worn. Each shoulder strap also is provided with an adjustable fastener connectable on the anterior surface of the wearer's shoulder between the wearer's trapezius muscle and the wearer's axilla, the fastener permitting the tightness or looseness of the shoulder loop to be adjusted by the wearer as desired while the brace is being worn. Each fastener is in the form of a hook and loop fastener with one of a hook fabric and a loop fabric being fixed to the shoulder strap in a position that overlies the anterior surface of the wearer's shoulder in the before mentioned area when the brace is being worn, and the other of the hook fabric and loop fabric fixed to the free end of the strap. The free end of the strap is passed through a closed loop link that permits the size of the shoulder loop formed by the strap, and thereby the tightness of the brace on the wearer, to be tightened or loosened simply by drawing the strap through the link in one direction or the other. When the desired tightness of the shoulder loop is achieved, the hook fabric and loop fabric are simply pressed together by the wearer on the anterior surface of the wearer's shoulder.

U.S. Pat. No. 6,315,747—Posture Aid Apparatus

A therapeutic posture aid apparatus designed to properly align all three curves of the spine, hold the shoulder girdle in a proper posture position, allow full range of motion of all joints in the back, neck, shoulders and arms, reminding the wearer to move his/her back, neck and shoulders into a proper posture to eliminate/relieve, back vain, stress and muscle fatigue.

U.S. Pat. No. 8,808,212—Elastic Shoulder Support Device for Training Mind and Muscles for Proper Posture

A shoulder support device includes a flexible elastic continuous ring band and a slide adjustably movable on the band that divides the band into a pair of loops defining arm receiving apertures that are cupped over the curved deltoid region of each shoulder of a wearer to pull the shoulders back and downward under tension to achieve a correct posture position. The slide is moveable to adjust tension distributed to the wearer's shoulders. When the slide is positioned midway between the shoulder blades at the back of the wearer, it provides equal distributed tension on both shoulders. Moving the slide more to the left or more to the right allows the wearer to selectively adjust which shoulder receives more tension.

### U.S. Publication No. 20030153855A1—Posture Apparatus

A posture system is made from a single strap and having opposing ends. At the opposing ends are fastening elements. In use, the user places the strap about the back of the user's neck “N” and, thereafter, crosses the strap 100 at a point on the user's back. During tension, the user fastens the strap in order to secure the strap to the user. The loops across the front of the shoulders pull the shoulders back or remind a user to pull the shoulders back for better posture and health.

U.S. Publication No. 20040034901A1—Personal Device Shoulder Harness System

A personal device shoulder harness system comprised of one continuous strip of material. Each end of this single continuous strip of material, preferably vegetable tanned leather for it's unique and user friendly qualities, has a hook an/or loop fastening fabric material of a suggested minimum 10% of the strips length or six inches in length, and as wide as the material used for the harness system, at each end to accommodate adjustments by the wearer. The hook and loop fastening fabric material closures are located to meet at the right front shoulder. This single continuous strip of material crisscrosses over the back of the wearer. There is an adjustment coupling apparatus where the one continuous strip of material crisscrosses over the back to provide adjustment by the wearer. And there will be a d-ring or facsimile apparatus where the user can attach their personal device of choice and, this apparatus will be attached to the one continuous strip of material by a rivet, or facsimile, which could create a permanent location, or an aluminum post with a screw, which could be relocated by the wearer to their liking, or facilitate the clanging of the d-ring to an alternative apparatus, or facsimiles of either of any number of available apparatuses. This one continuous strip of material is to be simple and durable in construction, inexpensive to manufacture, and will be efficient and useful to facilitate the wearer with a personal device shoulder harness system to attach their personal device[s] of choice.

### U.S. Publication No. 20120078149A1—Orthopedic Posture Brace

One embodiment of an orthopedic posture brace comprising a holster strap that forms two arm loops when attached to an axial strap and two abdominal straps. When worn, the holster straps encircle the user's shoulders. The axial strap will rest against the thoracic region of the user's back. After the two abdominal straps pass through a loop at the bottom of the axial strap, the two ends not connected to the holster strap may be fastened together around the front of the user's abdomen. Tightening the ends of the abdominal strap will cause tension in the holster straps urging the user's shoulders rearward to encourage proper spinal alignment and reduced slouching or hunching over. The amount of tension generated by tightening the abdominal straps will determine how far back toward the spinal column the user's shoulders are pulled.

Each of the references above has inherent disadvantages, including being overly complicated, expensive to manufacture, cumbersome to put on or take off or wear, or just being marginally effective. Therefore, it would be desirable to provide a shoulder compression harness that is simple in design, inexpensive to manufacture, and which effectively uses the elastic compression loops to place and hold the wearer's shoulders in proper position comfortably for long periods of time, so that the muscle memory eventually maintains the proper posture even when the shoulder compression harness is not being worn.

### SUMMARY OF THE INVENTION

The above objectives are accomplished according to the present invention by providing, in a first embodiment, a shoulder compression harness made from a stretchable material and formed essentially into a figure 8 configuration, wherein the intersection of the figure 8 where the material crosses over itself is fixed in place to provide a pair of stretchable loops. Each loop fits over a wearer's shoulders, and the compression material urges the shoulders rearwardly, in order to prevent slouching and to provide and maintain proper posture.

In a preferred embodiment, a handle member made from strap material is attached to a front portion of each loop, and a third handle strap is positioned on the back of the harness at the intersection of the stretchable material. These straps are used to allow the wearer to put on the harness and adjust it to the proper position, and further, the rear strap is used to take the shoulder harness off. In use, a wearer puts his or her arms through the loops and slides the intersection portion behind their head and onto their back. Then the user can grab either loop handle strap to get the harness situated on their body properly and comfortably. While the harness is being worn, the shoulders are urged backwardly to form proper posture, and the harness may be worn either during regular daily activities or during exercise sessions. When the wearer wishes to take the harness off, he or she may simply reach back and grab the rear handle strap and pull it upwardly over the wearer's head, and then slide the loops off of the shoulders, and down the arms for removal thereof.

### DESCRIPTION OF THE DRAWINGS

The invention will be more readily understood from a reading of the following specification and by reference to the accompanying drawings forming a part thereof, wherein an example of the invention is shown and wherein:

FIG. 1 shows a perspective view one embodiment of a shoulder compression harness in accordance with one aspect of the present invention;

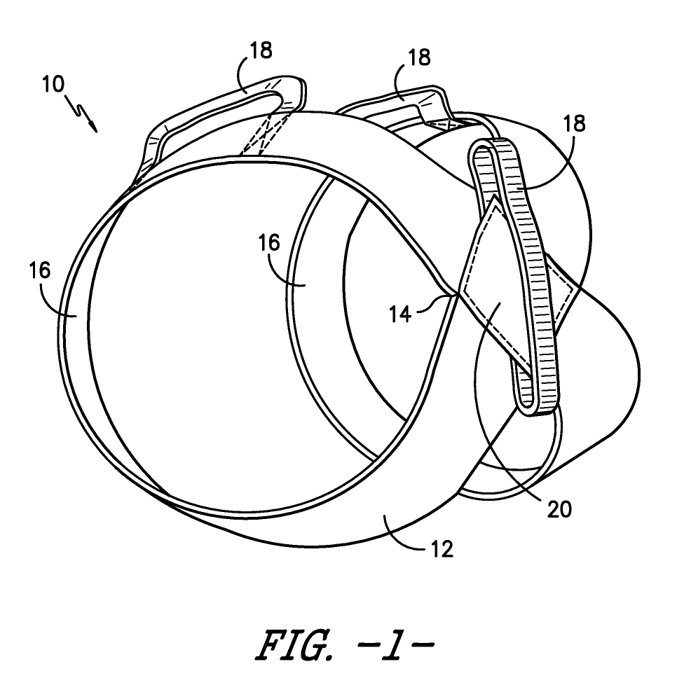

FIG. 2 is a front view of one embodiment of a shoulder compression harness positioned on a wearer; and

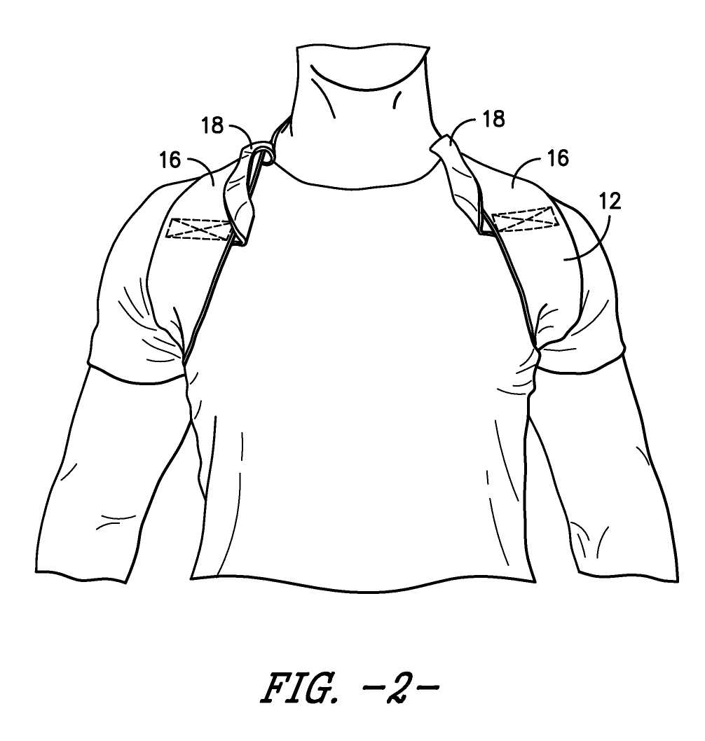

FIG. 3 is a perspective rear view of one embodiment of a shoulder compression harness positioned on a wearer.

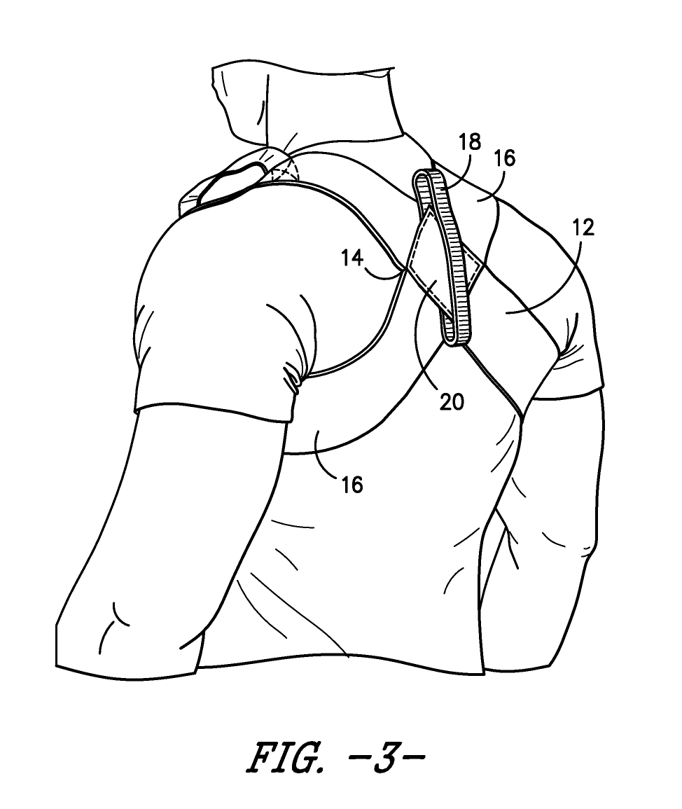

### DESCRIPTION OF A PREFERRED EMBODIMENT

Referring now to the drawings, the invention will be described in more detail. In a preferred embodiment, a shoulder compression harness 10 includes a single elastic strap 12 formed into a figure 8 configuration, as shown. The intersection of the elastic strap is affixed to itself at a hub 14, so that where the strap crosses itself at the hub 12, that intersection is fixed in position in any suitable manner (for example, by sewing, glue, or any other suitable method or combination of methods), in order to provide two generally equal size shoulder loops 16. The shoulder loops 16 are designed to fit over a wearer's shoulders, and each shoulder loop 16 includes a handle strap 18, or the like, on a front portion thereof, so that a wearer may adjust the position of each shoulder loop 16 around the shoulder so that it fits comfortably and provides proper posture. When the shoulder compression harness 10 is properly worn, the intersection, or hub 14, is disposed at a central portion of the wearer's back, between the shoulder blades.

In a preferred embodiment, the hub 14 also includes a handle strap 18 attached thereto. This rear handle strap 18 is useful for adjusting the hub 14 to the proper position when the wearer is putting on the shoulder compression harness 10, and is further useful for removing the harness 10.

To put on the compression harness 10, a wearer simply slides his arms through the shoulder loops 16, and then slides the hub 14 section over his or her head until the hub 14 reaches a central portion of the wearer's back, between the shoulder blades. The wearer can adjust the shoulder loops 16 by grabbing the front handle straps 18 on each side and adjusting the shoulder loops 16 until they are in proper position and are comfortable, and may adjust the back of the harness by grasping the rear handle strap 18 for appropriate adjustment.

The compression harness 10 may be used and worn during normal daily activities, and/or may be worn during exercise sessions. In either situation, the compression harness 10 urges the shoulders rearwardly into proper postural position, and the compression harness 10 may be worn over long periods of time, as desired. Eventually, the compression harness 10 trains the wearer's body, muscles, tendons, etc. to remain in the proper position, and muscle memory helps to maintain proper posture even when the compression harness 10 is not in place. During exercise, and especially during weightlifting activities, proper posture is important to achieve the best results, and is essential to avoiding injury. Thus, wearing the instant shoulder compression harness 10 during exercise provides additional health benefits and helps to reduce the chances of an injury.

In order to remove the harness 10, a wearer simply reaches back over his or her shoulder, grasps the rear handle strap 18, and pulls it upwardly over the wearer's head, and then slides the loops 16 down the wearer's arms until the harness 10 is removed. The front handle straps 18 and the rear handle strap 18 are preferably sewn directly onto the elastic strap using an “X in a box” seam, as shown, although any suitable attachment means may be used.

Optionally, a hub patch 20 may be sewn or otherwise attached to the intersection point of the elastic strap, as shown. The hub patch 20 adds strength to the intersection point, and further, advertising or logos may be printed on the hub patch 20.

One advantage to the present arrangement, wherein the elastic strap 12 is simply a loop folded over to form a figure 8 configuration, is that this arrangement provides compression in the proper direction for posture correction and maintenance, and further, it provides a structure that is more robust and stronger than other harnesses that include separate straps that are simply sewn together. Having a single loop formed into the figure 8 configuration means that the structural integrity of the harness 10 is not dependent upon sewn seams or small threads. In other words, even if a seam fails on the present invention, the structural integrity of the harness 10 remains intact.

Although the present invention has been described in considerable detail with reference to certain preferred versions thereof, other versions are possible. Therefore, the spirit and scope of the appended claims should not be limited to the description of the preferred versions contained herein. All features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

1. A shoulder compression harness comprising:
a single loop of elastic material, wherein said elastic material is formed into a figure 8 configuration so that a pair of equal sized shoulder loops is provided, and said elastic material crosses over itself in a central portion thereof, forming a hub;
wherein said elastic material is affixed to itself at said hub;
wherein each shoulder loop includes a handle member disposed on a front portion thereof, and
wherein said hub includes a handle member attached thereto;
to whereby said shoulder compression harness may be worn by a wearer so that said shoulder loops fit over said shoulders, and said hub may be positioned at a central portion of said wearer's back between said wearer's shoulder blades.

2. The shoulder compression harness set forth in claim 1, wherein said elastic material is affixed to itself at said hub by a method selected from the group of sewing, adhesive, and any combination thereof.

3. The shoulder compression harness set forth in claim 1, wherein said handle members are formed from straps.

4. The shoulder compression harness set forth in claim 1, further including a hub patch affixed to said hub.
